(19) United States
(12) Patent Application Publication
(10) Pub. No.: US 2022/0022778 A1
(43) Pub. Date: Jan. 27, 2022

(54) SYSTEM AND METHODS FOR PROVIDING PERSONALIZED WORKOUT AND DIET PLANS

(71) Applicant: BODYBUDDY ALGORITHMS INC., MONTREAL (CA)

(72) Inventor: TOMAS GAUTHIER, COWANSVILLE (CA)

(73) Assignee: BODYBUDDY ALGORITHMS INC., MONTREAL (CA)

(21) Appl. No.: 17/297,234

(22) PCT Filed: Nov. 15, 2019
(86) PCT No.: PCT/CA2019/051630

§ 371 Date: May 26, 2021

Related U.S. Application Data
(60) Provisional application No. 62/771,718, filed on Nov. 27, 2018.

(51) Int. Cl. A61B 5/11; A61B 5/107; A61B 5/00; G16H 20/60; G16H 20/30

(52) U.S. Cl. CPC A61B 5/1118; A61B 5/1079; G16H 20/30; G16H 20/60; A61B 5/0077

(57) ABSTRACT

An embodiment provides systems and methods for devising diet and fitness plans for a user, including an electronic device: receiving baseline measurement data relating to the user before a period of training and diet activity; receiving fitness goals data relating to the user; creating a training plan and a diet plan for the user based on the user's baseline measurement data, and fitness goals data; receiving data describing the user's diet and training behaviour during the period of training and diet activity; receiving current measurement data relating to the user after the end of the period of training and diet activity; comparing the baseline measurement data to the current measurement data; determining if the user's fitness goals were met based on the comparing the current measurement data to the baseline measurement data.

### CROSS-REFERENCE TO RELATED APPLICATIONS

This application is a national stage of International Application No. PCT/CA2019/051630 filed on Nov. 15, 2019, which claims priority to U.S. Provisional Patent Application No. 62/771,718 filed on Nov. 27, 2018, all of which are hereby incorporated by reference in their entireties.

### FIELD

The present invention relates to systems and methods for electronic health assessment and instruction. More specifically, the present invention relates to systems and methods for analyzing a user's fitness level and providing a flexible personalized diet and fitness plans based on the user's specific wants and needs. The personalized diet and fitness plans may consider the user's current fitness level, body type, diet and exercise preferences, ongoing behaviour, fitness goals, and other factors.

### BACKGROUND

Obesity is at an all-time high worldwide. Recent studies estimate 30% of the world population is overweight. It is estimated that roughly 1 in 3 adults in the United States, and 1 in 4 adults in Canada, are now living with obesity. Obesity is expected to reach 45% in the United States by 2030. With this rise in obesity many people are looking for more effective approaches to help them achieve better fitness.

Conventional approaches to fitness, such as personal trainers and nutritionists, can be very expensive. Training for 3 sessions a week with a personal trainer can cost as much as $12,000 annually. Annual nutrition assessments and weight management programs can cost as much as $2000.

Self-managed workout plans often prove ineffective since people tend to rely on off-the-shelf workout plans which do not account for their individual needs/goals or specific body types. Scales alone also fail to help people properly assess their own fitness since they typically do not measure body mass index (BMI), body fat percentage, muscle mass, or physical measurements.

Most people struggling with weight issues do not have access to professional fitness aids and those that do typically have to pay large amounts of money and schedule specific times with fitness professionals who don't often take individuality into account. Current approaches are more cost effective and time flexible but do not provide tailored plans or accurate objective fitness assessments.

Nearly 66% of overweight people are estimated to possess internet-connected smart phones or similar devices, however, current fitness related smart phone applications lack the effectiveness of a personally tailored workout plan.

Therefore, what is needed is methods and systems which use smart phones or other similar electronic devices to provide personalized diet and fitness plans which can accurately assess a user's level of fitness and provide diet and fitness plans which are tailored to the individual user's needs and preferences.

### BRIEF SUMMARY

It is contemplated that the present invention can provide methods and systems for electronically creating a personalized diet and fitness plan for a user based on a combination of user measurement data, user fitness goals, and user preferences. It is further contemplated that, after a period of training and diet activity, the systems and methods can evaluate how successful the diet and fitness plan was at helping the user reach his/her fitness goals and, if necessary, amend the personalized diet and fitness plan to better help the user reach his/her fitness goals.

In one embodiment, the present invention provides systems and methods of devising a diet plan and a fitness plan for a user over an electronic communication network, including the steps of, an electronic device receiving baseline measurement data relating to the user before a period of training and diet activity, the electronic device receiving fitness goals data relating to the user, the fitness goal associated with the baseline measurement data, assembling a training plan for the user based on at least one the baseline measurement data and the fitness goals data, the training plan selected from at least one modular training plan element retrieved by the electronic device from a central database, each at least one modular training plan element associated with at least one of the baseline measurement data and the fitness goals data, assembling a diet plan for the user based on at least one of the baseline measurement data and the user's fitness goals data, the diet plan selected from at least one modular diet plan element retrieved by the electronic device from a central database, each at least one modular diet plan element associated with at least one of the baseline measurement data and the fitness goals data, the electronic device receiving behavioural data describing at least one of the user's diet behaviour and the user's training behaviour during the period of training and diet activity, the electronic device receiving current measurement data relating to the user after the end of the period of training and diet activity, determining the difference between the current measurement data and the baseline measurement data to generate a measurement data difference, and comparing the measurement data difference to the fitness goal.

In another embodiment, the present invention provides systems and methods of devising a diet plan and a fitness plan further including the steps of, if the measurement data difference does not satisfy the fitness goal, the electronic device setting values of the baseline measurement data to values of the current measurement data, the electronic device setting the training plan to be an updated training plan, the updated training plan selected from at least one modular training plan element retrieved by the electronic device from a central database, each at least one modular training plan element associated with at least one of the baseline measurement data and the fitness goals data, the electronic device setting the diet plan to be an updated diet plan selected from at least one modular diet plan element retrieved by the electronic device from a central database, each at least one modular diet plan element associated with at least one of the baseline measurement data and the fitness goals data, the electronic device setting the period of training and diet activity to be an upcoming period of training and diet activity, the electronic device receiving updated behavioural data describing at least one of the user's diet behaviour and the user's training behaviour during the updated period of training and diet activity, the electronic device receiving updated current measurement data relating to the user after the end of the updated period of training and diet activity, determining the difference between the updated current measurement data and the baseline measurement data to generate an updated measurement data difference, and comparing the updated measurement data difference to the fitness goal.

In yet another embodiment, the user's diet and fitness plan can be customized based on at least one of: the user's exercise preferences, the exercise equipment available to the user, the user's dietary preferences, and the user's dietary restrictions.

In yet another embodiment, the user's measurement data (e.g., height, waist size, chest size, etc.) is determined by collecting visual data of the user (e.g., photographs, videos, etc.) and processing them using photogrammetry.

Finally, in yet another embodiment, the system may determine the probability of the user achieving his/her fitness goals and, if the user has a higher probability of failing his/her fitness goals than succeeding, the system may suggest fitness goals which the user has a higher probability of achieving.

### DESCRIPTION OF FIGURES

The present invention will be better understood in connection with the following Figures, in which:

FIG. 1 is a diagram of one embodiment of a method in accordance with the present invention;

FIG. 2 is a diagram of two example text input screens for one embodiment of a system in accordance with the present invention;

FIG. 3 is diagram of an example visual data input screen for one embodiment of a system in accordance with the present method;

FIG. 4 is a diagram of an example three-dimensional model body part selection screen for one embodiment of a system in accordance with the present invention;

FIG. 5 is a diagram of an example fitness goal input screen using a three-dimensional model for one embodiment of a system in accordance with the present invention;

FIG. 6 is a diagram of an example screen depicting a comparison between a user's current physical state the user's fitness goals using three-dimensional models for one embodiment of a system in accordance with the present invention;

FIG. 7 is a diagram of an example training plan output screen for one embodiment of a system in accordance with the present invention;

FIG. 8 is a diagram of an example diet plan output screen for one embodiment of a system in accordance with the present invention;

FIG. 9 is a diagram of a suitable user device for use in accordance with the present invention; and

FIG. 10 is a diagram of two suitable user devices in electronic communication over an electronic communication network in accordance with the present invention.

### DETAILED DESCRIPTION OF EMBODIMENTS

It is contemplated that the present invention can provide methods and systems for electronically creating a personalized diet plan and fitness plan for a user based on a combination of user measurement data, user fitness goals, and user preferences. It is further contemplated that, after a period of training and diet activity, the systems and methods can evaluate how successful the diet plan and fitness plan was at helping the user reach his/her fitness goals and, if necessary, amend the diet plan and fitness plan to better help the user reach his/her fitness goals. Furthermore, it is contemplated that the present invention can track successful diet plan and fitness plans so that users of the present invention can benefit from diet plan and fitness plans that are better calibrated to the individual user's requirements and measurements.

In the context of the present invention it will be appreciated that “electronically creating a personalized diet plan and fitness plan” means using an electronic device to collect and analyze user data and produce a “diet plan” and a “fitness plan” which is personalized to a particular user's wants and needs and it is contemplated that these diet plans and fitness plans can be produced faster and/or cheaper than consulting with a physical trainer, nutritionist or other similar professional.

Moreover, it is contemplated that “creating a personalized diet plan and fitness plan” means assembling modular diet plan elements and modular fitness plan elements to result in personalized diet plans and fitness plans. It is contemplated that suitable modular diet plan elements and modular fitness plan elements are stored in a central database and that each potential modular diet plan element and modular fitness plan element is associated with at least one of the user's fitness goals and the user's measurement data, as will be discussed in further detail herein.

For further clarity, the “central database” which contains the modular fitness plan elements and the modular diet plan elements may be stored locally on the electronic device or is electronically accessible via network communications. For example, contents of the central database may be stored on a local device or may be retrieved as needed from a cloud server, or any other suitable known methods.

It is contemplated that an “electronic device” may be any suitable electronic device that can perform data calculations and receive data directly from a user or indirectly from other electronic devices. Furthermore, the electronic device may be capable of communicating with other devices over an electronic communication network, where an “electronic communication network” can be any suitable electronic network, including, but not limited to, a local Wi-Fi network, a global internet network, or two electronic devices in suitable near field communication. In at least one embodiment it is contemplated that the user device includes a digital camera and/or means for obtaining a digital photograph.

In the context of the present invention it will be appreciated that user “measurement data” be any data which describes or correlates to the physical state of a user, including, but not limited to, age, weight, height, sex, and body dimensions (e.g., waist size, chest size, etc.). Measurement data may further require the user to perform an activity, such as requiring the user to lift increasingly heavy weights with a muscle group to determine the user's lifting limits, as will be readily understood by the skilled person. In some embodiments, it is contemplated that measurement data is associated with a user's fitness goals and further can be associated with at least one of the modular fitness plan elements and the modular diet plan elements. It is further contemplated that measurement data may be inputted directly by the user or obtained via visual imaging techniques, such as photogrammetry.

In some embodiments, it is contemplated that known photogrammetry techniques can be used to ascertain user measurement data from at least one photograph that is captured by the electronic device. In this way, user measurement data can be extrapolated from a first photograph and can be subsequently updated from further photographs. In this way, it is contemplated that a user can readily obtain and automatically input measurement data for use in connection with the present invention in order to assemble modular diet plan elements and modular fitness plan elements to result in personalized diet plans and fitness plans.

In the context of the present invention it will be appreciated that a user's “fitness goals” may be any desired state of the user's body to achieve or maintain, including, but not limited to, improve definition of at least one muscle group (e.g., more defined biceps), lose fat, improve aerobic or athletic performance, lose weight, or gain muscle mass.

Furthermore, it is contemplated that the “user fitness goals” may include a desired timeframe within which to reach the desired physical state (e.g., lose 10 pounds in 4 weeks). It is also contemplated that a user fitness goal may be associated with a user's measurement data and moreover is associated to at least one of a modular fitness plan element and a modular diet plan element.

In some embodiments, it is contemplated that certain fitness goals may be considered fundamentally incompatible and therefore it is further contemplated that the present method can account for this. For example, catabolic goals (i.e.: reduced fat levels) may be considered incompatible with anabolic goals (i.e.: weight gain), as will be understood by the skilled person.

Moreover, it is contemplated that in some embodiments a suitable fitness goal can have an associated priority that reflects the level of intensity that the user wishes to approach the fitness goals with. In some embodiments, it is contemplated that the associated priority can be reflected on a numerical scale. As such, the resulting fitness plan and diet plan devised by the present method can be calibrated to account for both the user fitness goals and, in some embodiments, an associated priority.

For example, if the user's fitness goals were to increase the size of the user's biceps, the system may consider the user's measurement data (i.e.: the current size of the user's biceps) and the system may retrieve at least one modular fitness plan element from the central database which is associated with bicep size, such as dumbbell bicep curls, and as such is associated, with in this embodiment, both the user's fitness goals (an increase in the size of the user's biceps) and the user's measurement data (the actual size of the user's bicep).

Similarly, it is further contemplated that modular diet plan elements are also associated with at least one of the user's fitness goals and the user's measurement data, as will be discussed in further detail below. For example, as high protein diets are typically correlated to the goal of gaining muscle mass, this may be taken into consideration when automatically devising a personalized diet plan for the user. Therefore, modular diet plan elements associated with an increase in muscle mass (i.e.: a high overall daily caloric intake, regular servings of lean meat and eggs, X grams of protein per day, etc.) may be retrieved from the central database. As such, these modular diet plan elements can be considered associated with the user's fitness goals (increase the size of the user's biceps) and the user's measurement data (the actual size of the user's biceps).

In the context of the present invention it is contemplated that a “fitness plan” can be a schedule of physical training activity including at least one modular fitness plan element and a “diet plan” can be instructions for nutritional activity including at least one modular diet plan element.

In the context of the present invention it will be appreciated that “modular fitness plan elements” may be any exercise technique or physical movement of the user's body, including, but is not limited to, aerobic/endurance exercises (e.g., running, biking, swimming, hiking, etc.), strength exercises (e.g., weight lifting, push-ups, resistance training, etc.), flexibility exercises (e.g., yoga, stretches, etc.), balance exercises (e.g., Tai Chi, balancing poses, etc.) and hybrid exercises (playing team sports, CrossFit, etc.). Furthermore, it is contemplated that these exercises may or may not involve the use of specialized exercise machinery or other equipment.

Moreover, it is contemplated that a “modular fitness plan elements” may range from strictly scheduled to very flexible. For example, a strict schedule may be “everyday at 3:00 pm walk at a pace of 5 km/h for 45 minutes” which includes a set day (everyday), a set time (3:00 pm), a set activity (walk), a set intensity (5 km/h), and a set duration (45 minutes). An example flexible schedule may be “perform 1 hour of aerobic exercise 5 days a week”, allowing the user to decide which days, which times, which activities, and what levels of intensity. For clarity, none of the elements: day, time, activity, intensity, or duration are required, allowing the personalized workout and diet system and methods to be as specific or flexible as desired for each particular user.

In some embodiments, it is contemplated that suitable modular fitness plan elements can be categorized based on the type of physiological response that is encouraged by each particular modular fitness plan element. For example, in some embodiments it is contemplated that suitable modular fitness plan elements are categorized as “endurance”, “interval”, and “weight lifting”. Other possible categories include, but are not limited to, “aerobic”, “anaerobic”, “catabolic”, “anabolic”, “stretching”, “low impact” and “high impact” as will be readily understood by the skilled person.

For example, modular fitness plan elements categorized as “endurance” could be considered exercises which are aerobic and which use fat as a primary fuel source. Typically, “endurance” modular fitness plan elements are not associated with mass gain/anabolic fitness goals and rather are associated with fat loss/catabolic fitness goals, as will be readily appreciated by the skilled person.

Modular fitness plan elements categorized as “interval” could be considered exercises that are a mix of aerobic and anaerobic activity (i.e.: soccer, hockey, football, circuit training and CrossFit® and team sports). Typically, these types of “interval” modular fitness plan elements are successful at emptying glycogen stores and ensuring that any consumed carbohydrates is redirected for glycogen replenishment rather than fat storage as will be understood by the skilled person. Interval modular fitness plan elements would fall between fat loss and muscle gain and as such can be associated with a wide variety of fitness goals. As will be understood by the skilled person, interval modular fitness plan elements recruits mostly Type I (aerobic) and Type Ila (glycolytic anaerobic) muscle fibers, as will be readily appreciated by the skilled person.

Modular fitness plan elements categorized as “weight training” can be characterized as periods of high intensity exercise with periods of rest while recruiting all muscle fibers, including powerful Type Ilb muscles. While weight training modular fitness plan elements can be associated for all fitness goals, it is emphasized for mass gain/anabolic fitness goals.

In the context of the present invention it will be appreciated that “modular diet plan elements” may be any activity by a user which involves the consumption of food or drink, including but not limited to, meals or snacks eaten, drinks consumed, and vitamin supplements taken.

In the context of the present invention it will be appreciated that “modular diet plan elements” may range from a strictly scheduled meal plan for all food and drink consumed during a period of diet activity (e.g., Monday at 8:00 am for breakfast have a 70 gram bowl of oatmeal, a 200 ml glass of orange juice, and large two eggs, etc.) to much more flexible instructions (e.g., for each meal consume 70 grams of carbohydrates, 50 grams of protein, and 15 grams of fat). For clarity, none of these elements, including but not limited to, day, time, meal, specific food or drink, specific amount of food or drink, or amount of macronutrients (carbohydrates, protein, fat) are required, allowing the personalized workout and diet systems and methods to be as specific or as flexible as desired for each particular user. Furthermore, the “modular diet plan element” may include instructions for the user to take vitamin supplements (e.g., vitamin C, B complex, etc.) or other supplements.

In the context of the present invention it will be appreciated that “user preferences” may be any preferred approach to physical training activity or nutritional activity, including, but not limited to, which exercise machines are preferred or available (e.g., does not have access to a gym, has access to a stationary bicycle, etc.), which activities to prefer or avoid (e.g., does not want to run, likes to swim, etc.), or which foods and drinks are preferred or available (e.g., allergic to peanuts, likes bananas, etc.). For clarity, it is not required to provide any one or any combination of the above (e.g., the user can specify only preferences and not things to avoid or can specify nothing at all). Any preferences provided allow the personalized workout and diet system and methods to customize the fitness plan to better suit the user.

In the context of the present invention it will be appreciated that “a period of diet and training activity” may be any period of time during which the user is expected to be following some instructions for physical training activity or instructions for nutritional activity (e.g., a day, a week, a month, etc.).

In the context of the present invention it will be appreciated that “how successful the fitness plan was at helping the user reach his/her fitness goals” is a determination made by the personalized workout and diet methods and systems as to whether the user is making sufficient progress towards his/her fitness goals such that when the desired period of time has elapsed, the user will have met or exceeded his/her fitness goals. This determination may be made by any algorithmic evaluation criteria, including, but not limited to, calculating a mathematical equation which models the rate of the user's progress and predicting what the user will have achieved at the end of the desired period of time, based on extensions of the mathematical equation; and tracking large scale trends across many users and adjusting future predictions based on applied machine learning techniques. For clarity, “successfulness” is contemplated as an objective criterion based on an algorithmic model's predicted future state of the user.

In at least one embodiment, it is contemplated that the user's success will be determined based on a comparison of the user's current measurements and the user's baseline measurements, and subsequently comparing this determined difference (if any) to the user's fitness goals.

In this way, it is contemplated that, in some embodiments, fitness goals will be directly correlated to measurement data for ease of evaluation, as discussed above. For example, in the simplest case, the user's fitness goal may be to lose 10 pounds. Whether the user has succeeded in losing 10 pounds can be determined by calculating the difference between the user's previous weight and the user's current weight and checking if that difference is greater than or equal to −10. Goals such as improving biceps may be evaluated by comparing the previous size of the user's biceps to the current size of the user's biceps or comparing the user's previous bicep weight lifting limit to a new bicep weight lifting limit. When the difference between the user's baseline measurement and current measurement meets or exceeds the fitness goals' desired difference, the fitness goals will be deemed satisfied.

Moreover, it is further contemplated in at least one embodiment that at least one of the fitness goals and the measurement data is further associated with a modular fitness plan element and a modular diet plan element. In this way and as discussed herein, a diet plan can be assembled of modular diet plan elements that are associated with at least one of the user's fitness goals and the user's measurement data. Similarly, it is contemplated that a fitness plan can be assembled of modular fitness plan elements that are associated with at least one of the user's fitness goals and the user's measurement data.

### Example 1

Fitness Goal: FAT LOSS

Priority: Low
Modular diet plan elements: 10-15% caloric deficit. Macronutrient ratio: P40% F20% C40%
Modular fitness plan elements: Weight training (complex exercises) 2×/wk for 30 min. Aerobic exercise 3-4×/wk for 20 min.

Priority: Medium
Modular diet plan elements: 15-20% caloric deficit. Macronutrient ratio: P50% F20% C30% Modular fitness plan elements: Weight training (complex+isolation exercises) 2-3×/wk for 45 min. Aerobic exercise 3-4×/wk for 30-45 min. Interval training 1-2×/wk for 30 min.

Priority: High
Modular diet plan elements: 25% caloric deficit. Macronutrient ratio: P20% F70% C10%
Modular fitness plan elements: Weight training (complex+isolation exercises) 2-3×/wk for 60 min. Aerobic exercise 4-5×/wk for 45-60 min. Interval training 1-2×/wk for 30-45 min.

### Example 2

Fitness Goal: MASS GAIN

Priority: Low
Modular diet plan elements: 5-10% caloric surplus. Macronutrient ratio: P30% F20% C50%
Modular fitness plan elements: Weight training (complex exercises+isolation−focus on eccentric) 3×/wk for 45 min. Aerobic exercise after weight training for 20 min.

Priority: Medium
Modular diet plan elements: 10-15% caloric surplus. Macronutrient ratio: P25% F15% C60% Modular fitness plan elements: Weight training (complex exercises+isolation−focus on eccentric) 4-5×/wk for 45-60 min. Aerobic exercise after weight training for 20 min.

Priority: High
Modular diet plan elements: 15-20% caloric surplus. Macronutrient ratio: P30% F10% C60%
Modular fitness plan elements: Weight training (complex exercises+isolation−focus on eccentric) 6-7×/wk for 60 min., including active recovery. Aerobic exercise after weight training for 20 min.

As will be readily appreciated by the skilled person, it is contemplated that the system may rely on machine learning techniques which are known to one skilled in the art to weight both modular diet plan modules and modular fitness plan modules based on at least measurement data contexts. A “measurement data context” may be any combination of one or more pieces of measurement data which are found to correlate to a fitness goal outcome. For example, if, by tracking data from a number of users over time, it is found that a threshold ratio between two measurements frequently corresponds to a particular exercise being especially effective for a particular muscle group, this exercise may be suggested more often for users that demonstrate the threshold ratio compared to other users.

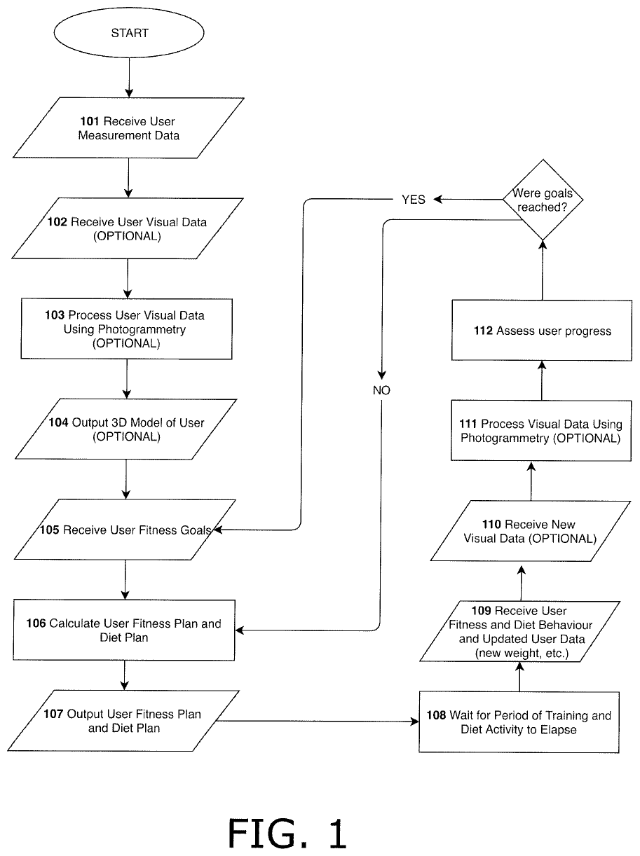

With reference to FIG. 1, a diagram of at least one embodiment of a method of electronically creating a personalized diet plan and fitness plan for a user based on a combination of user measurement data, user fitness goals, and user preferences is shown.

In this embodiment, the system is suitably configured to receive data relating to a user. It is contemplated that this step may include, but is not limited to, receiving inputs directly from a user via an interface presented to the user, or receiving pre-collected data from an external electronic device via network communication. First, the system receives the user measurement data 101.

Next, it is further contemplated that the system may additionally receive visual data relating to a user 102, which may include further user measurement data. It is contemplated that this step is optional in some embodiments and visual data may be omitted entirely from the method where the user's measurement data can be acquired through other means. In embodiments which receive visual data, the visual data can then be processed into a three-dimensional model of the user's body using photogrammetry 103 using known methods. Once a three-dimensional model has been calculated, the model can then be outputted to the user 104. The outputted model may be used as part of a fitness goal input means or may be outputted merely for the user to better visualize his/her own fitness level. In some embodiments, it is contemplated that the user's somatotype and weight distribution may be automatically determined from the resultant three-dimensional model, as will be readily understood by the skilled person.

Next, the system receives the user's fitness goals 105. Fitness goals may be received from the user directly via some input means, may be acquired from another electronic device via network communication, retrieved from a predetermined list or may be received by any other suitable means.

Once the system has received both the user measurement data at steps 101/102 and the user fitness goals at step 105, the system may calculate a training plan and a diet plan for the user 106. The fitness plan and diet plan are then outputted to the user by any suitable means 107. It is contemplated that the fitness plan and diet plan may be predetermined and retrieved from a centrally accessible database, while in other embodiments it is contemplated that the fitness plan and diet plan may be customized based on the needs and data obtained from a particular user. It is also contemplated that the fitness plan and diet plan may be a combination of predetermined and customized fitness plans and diet plans, as will be readily understood by the skilled person.

In at least one embodiment, it is contemplated that the fitness plan is constituted of at least one modular fitness plan element that is retrieved from a centrally accessible database. In these embodiments, it is contemplated that the modular training plan element is associated with at least one of the fitness goals and the user measurement data.

Moreover, in at least one embodiment it is contemplated that the diet plan is similarly constituted of at least one modular diet plan element that is retrieved from a centrally accessible database. In these embodiments, it is contemplated that the modular diet plan element is associated with at least one of the fitness goals and the user measurement data.

Once the compiled training plan and diet plan have been outputted to the user, the system waits for the necessary amount of time to elapse for the user to perform the training plan and diet plan 108. At this stage, the system may keep track of how much time has elapsed using an internal clock, receiving date information from an external electronic device via network communication, receiving an input from the user indicating that the period of time has elapsed, or by any other suitable means of time tracking.

After the necessary time period has elapsed, the system can receive new measurement data relating to the user and data describing the user's training and diet behaviour during the time period 109.

The new measurement data and user behaviour data may be received directly from the user via a user interface, may be received from an external electronic device via network communication, may be determined by the system based on information which is tracked by the system independently, or may be received by any other suitable means.

At this stage, there are again some embodiments where the system may receive new visual data 110 that includes user measurement data and process that visual data into a new three-dimensional model of the user's body 111. These steps are not required in all embodiments and user body dimensions including user measurement data may be acquired by any suitable means, including, but not limited to, being received with the other measurement data during previous step 109. In at least one embodiment, it is contemplated that at least one photograph can be obtained by the user using an electronic device and the new measurement data can be extracted from this photograph by known photogrammetry techniques, as will be readily appreciated by the skilled person.

Once the system has acquired the necessary user measurement data to determine the user's present state of fitness, the system compares the user's present state of fitness to the user's previous state of fitness and determines whether the user has reached or is making sufficient progress towards the user's fitness goals 112. If the user has reached his/her fitness goals, the system returns to step 105 and prepares to receive new fitness goals. If the user has not reached his/her fitness goals, the system returns to step 106 and recalculates a training plan and a diet plan for a new cycle based the user's fitness goals, current rate of progress, and measurement data. As discussed previously, it is contemplated that the previous fitness plan and diet plan may be incrementally adjusted. Alternatively, the training plan and fitness plan may be predetermined and retrieved from a centrally accessible database, while in other embodiments it is contemplated that the fitness plan and diet plan may be redesigned based on the needs and data obtained from a particular user. It is also contemplated that the fitness plan and diet plan may be a combination of predetermined and customized fitness plans and diet plans, as will be readily understood by the skilled person.

In at least one embodiment, it is contemplated that the recalculated fitness plan is constituted of at least one modular fitness plan element that is retrieved from a centrally accessible database and the recalculated diet plan is similarly constituted of at least one modular diet plan element that is retrieved from a centrally accessible database, in an analogous manner as discussed above.

As will be appreciated by those skilled in the art, the methods are contemplated to be perpetual. If a user reaches his/her fitness goals, then the user may enter new fitness goals and continue the process. If the user does not reach his/her fitness goals, the system considers any new information about the user, and the user's progress obtained in the previous cycle and repeats or refines the user's training plan and diet plan for another cycle.

### Data Input

In at least one embodiment, it is contemplated that the personalized workout and training system and methods will receive data pertaining to a user. This data may include user measurement data, user fitness goals, and user preferences (all of which have been defined in more detail above).

In at least one embodiment, the data may be received directly from the user through some means of user input. User input means may include, but is not limited to, the use of a keyboard, mouse, touchscreen, microphone, and/or camera or any other user device functionality.

In at least one embodiment, the user is presented with a series of text boxes, drop down menus, or other similar graphical user interfaces (GUI) which permit the user to enter any data required by the system. For example, the user may enter his/her sex, age (or date of birth), weight, height, fitness goals, fitness preferences/limitations, and dietary preferences/limitations. The user may be able to update the entered data as desired (e.g., new fitness preferences, new dietary preferences, new fitness goals, etc.) or as requested (e.g., enter weight after a period of training, etc.). Furthermore, the user may be requested to provide new data required by the system (e.g., enter the actual food which the user ate during a period of dietary activity, enter the actual exercise performed during a period of training activity, etc.).

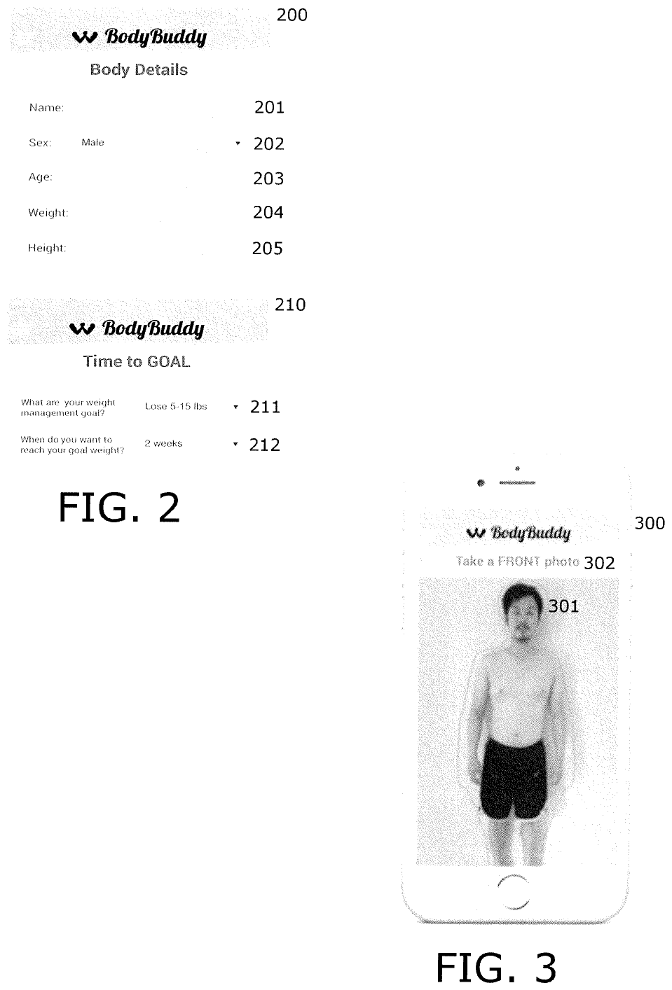

Two sample GUI input screens for use with an embodiment of the of the present invention are shown in FIG. 2. FIG. 2 depicts a Measurement Data Screen 200, and a Fitness Goals Screen 210. Measurement Data Screen 200 comprises a series of input boxes for the user to enter his/her measurement data using a mouse, touchscreen, connected keyboard, on-screen keyboard, microphone or other suitable input means. Sample Measurement Data Screen 200 depicts a Name Input Box 201, a Sex Input Dropdown Box 202, an Age Input Box 203, a Weight Input Box 204, and a Height Input Box 205. Sample Fitness Goal Screen 210 includes a Weight Management Goal Dropdown Box 211, and a Goal Timeline Dropdown Box 212.

It will be understood by those skilled in the art that FIG. 2 merely depicts example input screens which are suitable for the use in accordance with the present invention. Other methods of acquiring necessary data are possible without departing from the scope of the present invention. Other input methods may include, but are not limited, to receiving data from an external source via network communication or determining required data by analyzing other related data (e.g., determining a user's height by analyzing a photograph of the user). User data may further be collected automatically through a monitoring device. For example, the system may track the number of steps taken or calories burned by the user using some variety of wearable person devices or fitness trackers generally known to one skilled in the art.

In at least one embodiment, the system may receive visual data (e.g., photographs, videos, three-dimensional models, etc.) depicting the user's physical dimensions as discussed herein. The visual data may be received directly from the user via a camera connected to the electronic device (e.g., the user gets someone else to take photographs of the user from multiple different angles—front, back, side, etc.—and allows the system to access the photographs through the electronic device's internal storage). The visual data may also be acquired using an external means and transferred to the electronic device's internal storage or imported by the system from an external storage location (e.g., memory card, Dropbox® cloud storage, Google® cloud storage, etc.).

A sample visual data input screen for use with an embodiment of the present invention is depicted in FIG. 3. FIG. 3 depicts a Screen Layout 300 with an image of a User 301 currently visible with a connected camera and Instructions 302 for which type of photograph is required.

In at least one embodiment, the system may display a three-dimensional model of the user's body and provide the user with a means to select different parts of the three-dimensional body which the user wants to work on. The user may also be provided with a means to adjust parts of the three-dimensional model to input how they want their own body to look as a fitness goal.

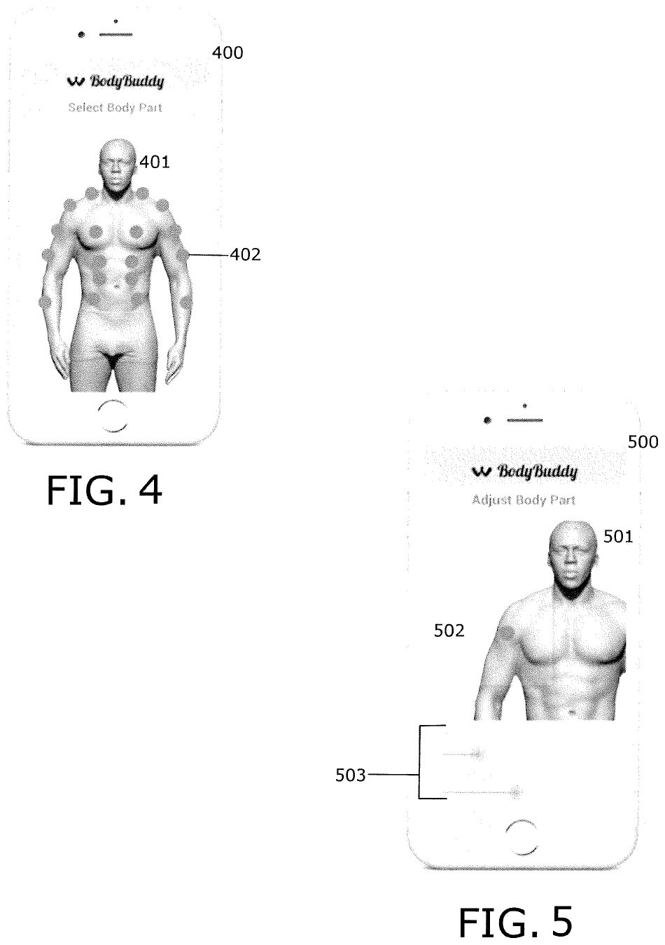

FIG. 4 and FIG. 5 depict sample input screens for interacting with a three-dimensional model. FIG. 4 depicts a Body Part Selection Screen 400 which displays a Three-Dimensional Model 401 and provides Interaction Points 402 which the user may select to indicate for which body parts the user wants to provide fitness goals. FIG. 5 depicts a Fitness Goal for Body Part Input Screen 500. The Fitness Goal for Body Part Input Screen 500 displays a Model Close-Up 501, a Highlighted Body Part 502, and Sliders 503 which the user can adjust to set fitness goals.

As will be understood by those skilled in the art, FIGS. 4-5 merely depict examples of how a three-dimensional model may displayed to a user and used for user inputs. Other methods of displaying or interacting with three-dimensional models are possible without departing from the scope of the invention.

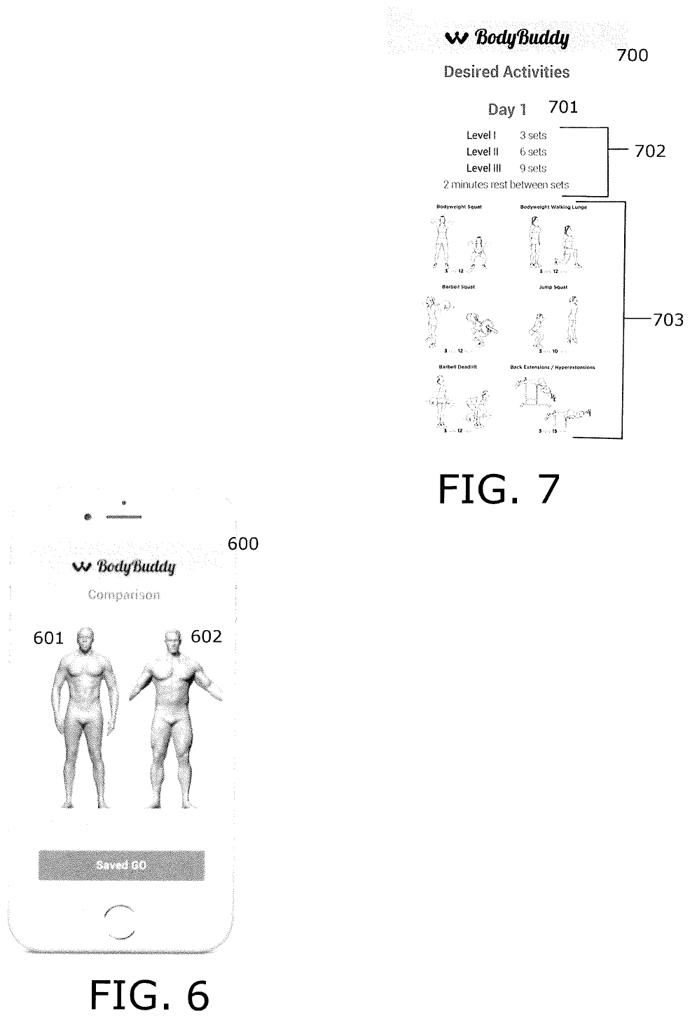

In at least one embodiment, once the system has acquired the user's fitness goals the system may display a comparison of the user's current fitness level against the user's desired fitness level. FIG. 6 depicts a sample Comparison Screen 600 with a Before Model 601 displayed next to an After Model 602. In at least one embodiment, once the fitness goals comparison has been displayed to the user, the user may confirm the fitness goals or may return to an earlier step to set new fitness goals.

### Photogrammetry

In at least one embodiment and as discussed herein, the system may use known methods of photogrammetry (the process of calculating the measurements of three-dimensional objects from two-dimensional images) to extract and calculate the user's body dimensions (e.g., waist size, chest size, etc.). The electronic device may calculate the user's body dimensions using its internal processing capabilities or may send the data to a more powerful computation device using network communication and receive the calculated results from the more powerful computation device once the dimensions are determined.

The photogrammetry analysis may use known trends in human body composition to reduce the processing requirements as will be readily understood by the skilled person. For example, patterns regarding the correlation of one body part measurement to another may allow the photogrammetry analysis to make assumptions which compensate for insufficiently clear (or incomplete) visual data. In some embodiments, it is contemplated that the user's somatotype and weight distribution may be automatically determined from the resultant three-dimensional model, as will be readily understood by the skilled person.

### Training Plan

In at least one embodiment, the system may use at least one of: user measurement data, user preferences, and user fitness goals to create a customized fitness plan that is comprised of associated modular training plan elements.

In at least one embodiment, the system may determine user measurement data by analyzing the user's body dimensions and sex to determine the user's body fat percentage. For example, in at least one embodiment the system may calculate the user's body fat percentage based on the US Navy Method. The US Navy Method requires different considerations for each sex. For male users, the system may require the user's height, neck circumference, and waist circumference (at navel). For female users, the system may require the user's height, waist circumference (narrowest point), and hips circumference (widest point).

According to the US Navy Method, once the system has received the required information, it is contemplated that the system may calculate the user's body fat percentage according to a fixed formula for each sex. For female users, it is contemplated that the body fat percentage may be determined by the formula:

Body Fat %=163.205*Log 10(Waist Circumference+Hips Circumference-Neck Circumference)−97.684*Log 10(Height)−78.387

For male users, the body fat percentage may be determined by the formula:

Body Fat %=86.010*Log 10(Waist Circumference−Neck Circumference)−70.041*Log 10(Height)+36.76

It will be understood by those skilled in the art that the US Navy Method is merely an example of one way by which a user's body fat percentage may be estimated. The skilled person will appreciate that body fat percentage may be estimated using a variety of different methods, any of which may be used without departing from the scope of the present invention.

In at least one embodiment, the system may further consider a variety of received or determined measurement data relating to a user to assess the user's present state of fitness. This measurement data may include, but is not limited to, body fat percentage, weight, shoulder girth, waist girth, hip girth, bicep girth, thigh girth, glute girth, and waist to hip ratio. It will be understood by those skilled in the art that these are merely examples of the types of measurements which may be used to assess a user's fitness and all, some, or none of these measurements may be used in combination with other measurements not listed herein without departing from the scope of the present invention.

In at least one embodiment, once the user's present state of fitness has been determined, the system may consider the user fitness goals (e.g., spot reduction of some area of the body, hypertrophy, etc.) to determine measurement data which would be required to meet the user fitness goals. The system may further suggest or enforce adjustment to the user fitness goals in order to reach desired measurement data that is more realistic (e.g., if the user's goals are physically impossible, unhealthy, or likely to fail, etc.).

In at least one embodiment, once the system has acquired the desired measurement data and fitness goals for the user, the system may consider various modular training plan elements which are available to and/or preferred by the user and associated with at least one of the user fitness goals and the measurement data. As discussed previously, it is contemplated that modular training plan elements may be predetermined and retrieved from a centrally accessible database, while in other embodiments it is contemplated that the modular training plan elements may be customized based on the needs and data obtained from a particular user. It is also contemplated that the modular training plan elements may be a combination of predetermined and customized training methods, as will be readily understood by the skilled person.

For example, the system may consider which modular training plan elements exist and are available to the user from an modular training plan element database (optionally cross-referenced with user preferences if applicable). Available modular training plan elements may include, but are not limited to, free weights, exercise machines, resistance cables/bands, bodyweights, sports, and sports-specific machines. For clarity, the system may provide suggestions for modular training plan elements which range from very specific (e.g., workout on a specific exercise machine) to very flexible (e.g., strength training).

In some embodiments, suitable modular training plan elements may further include recovery or therapy activities for the user to engage in during the period of diet and training activity. For example, based on a database of recovery and therapy activities (optionally cross-referenced with user preferences if applicable), available recovery and therapy activities may include, but are not limited to, thermotherapy, meditation, a sleep schedule, active recovery methods, or self-assessment exercises.

Based on the modular training plan elements, and recovery and therapy activities selected for the user (which may be none in any of the above categories), the system may prescribe schedules for each activity, including, but not limited to, the frequency of the activity (e.g., which days, how many times a day, etc.), the intensity (e.g., how much weight to lift, how fast to run, how long to run, etc.), the timing (e.g., which time of day to perform the activities, etc.), the types of activities to perform (e.g., machine exercises, callisthenics, etc.). For clarity, none of the above factors (either specific or flexible) are required and any of the above details may be omitted to better tailor a schedule of physical activity for a user.

An example output screen for a training plan is depicted in FIG. 7. FIG. 7 depicts an Training Output Screen 700 with the Day 701 on which the modular training plan elements should be performed, the instructed Intensity 702 of the modular training plan elements, and the Exercises 703 which include simple instructional images.

As will be understood by those skilled in the art, FIG. 7 merely depicts an example of how a training plan may be described and communicated to a user. Other methods of describing and communicating a training plan to a user are possible without departing from the scope of the invention.

Nutrition Planning

In at least one embodiment, it is contemplated that the system may use at least one of: user measurement data, user preferences, and user fitness goals to create a customized diet plan that is comprised of associated modular diet plan elements.

As discussed previously, it is contemplated the nutrition activity may be predetermined and constitute modular diet plan elements retrieved from a centrally accessible database, while in other embodiments it is contemplated that the nutrition activity may be customized based on the needs and data obtained from a particular user. It is also contemplated that the nutrition activity may be a combination of predetermined and customized nutrition activity, as will be readily understood by the skilled person.

In at least one embodiment, the system may obtain user measurement data by analyzing the user's body dimensions and sex to determine the user's basal metabolic rate (BMR). As will be understood by those skilled in the art, BMR is amount of energy per unit of time that a person uses/needs to keep their body functioning at rest. The system may calculate the user's BMR based on the Harris-Benedict Equation. The Harris-Benedict Equation requires different considerations for each sex. For both male and female users, the system may require the user's height, weight and age.

According to the Harris-Benedict Equation, once the system has received the required information, the system may calculate the user's BMR according to a fixed formula for each sex. For female users, it is contemplated that the BMR may be determined by the formula:

BMR=655.1+(4.35*Weight in lbs)+(4.7*Height in inches)(4.7* Age in years)

For male users, the BMR may be determined by the formula:

BMR=66+(6.2*Weight in lbs)+(12.7*Height in inches)−(6.76*Age in years)

It will be understood by those skilled in the art that the Harris-Benedict Equation is merely an example of one way by which a user's BMR may be estimated. The skilled person will appreciate that BMR may be estimated using a variety of different methods, any of which may be used without departing from the scope of the present invention.

In at least one embodiment, once the system has acquired user measurement data in the form of the BMR for a user, the system may use this to further acquire the total daily energy expenditure (TDEE) for the user. TDEE may be determined by multiplying the BMR by a number representing the user's physical activity level (PAL). The number scale used to represent a user's PAL may vary based on how different levels of physical activity are defined. For example, the system may use the scale: sedentary=1.4; moderately active=1.7; very active=2.0; and extremely active=2.4.

As will be understood by one skilled in the art, the above scale relies on the user's and/or system's understanding of where the lines between any of the above levels exist and may vary based on differing definitions of the terms (i.e., exactly how active is moderately active). As a result, it is contemplated that a scale of PAL which differs from the above based on different terms and divisions may be substituted without departing from the scope of the invention.

In at least one embodiment, once a user's TDEE has been acquired, the system may use the TDEE to determine a new suggested TDEE based on the user's fitness goals.

For example, if the user's fitness goal is to gain muscle mass, the system may consider varying degrees of mass gain diets, including, but not limited to, cautious mass gain (+5%; TDEE+(TDEE*0.05)), textbook mass gain (+10%; TDEE+(TDEE*0.10)), or aggressive mass gain (+15%; TDEE+(TDEE*0.15)).

If, on the other hand, the user's fitness goal is to lose fat, the system may consider varying degrees of fat loss diets, including, but not limited to, suggested fat loss (−15%; TDEE−(TDEE*0.15)), aggressive fat loss (−20%; TDEE−(TDEE*0.20)), or reckless fat loss (−25%; TDEE−(TDEE*0.25)).

As will be understood by one skilled in the art, the above approaches to fitness goals such as fat loss and mass gain are merely intended to be examples of the types of calorie adjustment approaches that may be used. The above examples are not intended to be limiting. Any other suitable calorie adjustment approaches may be used without departing from the scope of the invention. Furthermore, other fitness goals, such as body transformation, may be available, which may provide calorie adjustment approaches similar to or completely different from the examples given above.

In at least one embodiment, once the desired fitness goal and measurement data is acquired by the system, it is contemplated that the system may determine associated modular diet plan elements, In one embodiment, it is contemplated that the modular diet plan elements are in the form of macronutrient (type of food—e.g., fat, protein, carbohydrate—required in large amounts in the human diet) proportions for the user. As such, the system may determine a diet plan including suggested macronutrient ratio for the user based on the user's fitness goals.

For example, a user whose fitness goal is to lose fat may be advised to consume modular diet plan elements in the form of a diet of 20% carbohydrates, 40% protein, and 40% fat. A user whose fitness goal is to gain mass may be advised to consume modular diet plan elements in the form of a diet of 50% carbohydrates, 30% protein, and 20% fat. A user whose fitness goal is to transform his/her body may be advised to consume modular diet plan elements in the form of a diet of 30% carbohydrates, 40% protein, and 30% fat. As will be appreciated by one skilled in the art, these ratios are merely meant to serve as examples and ratios which differ from these for similar or different fitness goals may be used without departing from the scope of the invention.

In at least one embodiment, once the system has acquired associated modular diet plan elements in the form of a macronutrient ratio for the user, the system may further convert the macronutrient ratio into an amount (e.g., grams, ounces, etc.) of each macronutrient. In one embodiment, the system may convert from a ratio to a weight value by multiplying the TDEE by the relevant percent, divided by the conversion rate for each macronutrient. Conversion rates for macronutrients may be: carbohydrates=4 calories/gram; protein=4 calories/gram; and fat=9 calories/gram. In one embodiment, the conversion for macronutrients may be represented by the formulas:

Carbohydrates (grams)=TDEE*%/4

Protein (grams)=TDEE*%/4

Fat (grams)=TDEE*%/9

For example, if the macronutrient ratio determined for the user is 20% carbohydrates, 40% protein, and 40% fat then the suggested macronutrient amounts for the user would be: TDEE*0.2/4 grams of carbohydrates per day; TDEE*0.4/4 grams of protein per day; TDEE*0.4/9 grams of fat per day.

As will be understood by one skilled in the art, the above formulas and conversion rates are merely intended to be exemplary of a process that could be used to determine the appropriate amounts of macronutrients for a user. Accordingly, other known methods which produce similar results may be used without departing from the scope of the invention.

In at least one embodiment, once the amounts of macronutrients are acquired for the user, those amounts may be further divided among the number of meals, snacks, and/or drinks consumed by the user to provide more precise nutritional instructions. The amounts of macronutrients may be divided equally among the consumption activities or divided unevenly to suit the user's schedule and needs.

In at least one embodiment, once the amounts of macronutrients are acquired for a user, those amounts may be used to create a detailed diet plan for the user, also known as macros to meals. A detailed diet plan may be comprised of recipes or instructions for which things to consume throughout each day. Recipes to use for a diet plan may form part of the system or may be acquired from external databases as needed. For example, recipes may be retrieved from USDA National Nutrient Database, Canadian Nutrient Files, CIQUAL French Food Composition Tables, Souci-Fachman-Kraut Food Composition and Nutrition Tables, Food Composition Tables of Greek Foods and Greek Dishes, Banca Dati di Composizione delgi Aliomenti, McCane and Widdowson's The Composition of Foods Integrated Dataset, Swiss Food Composition Database, NFA Food Composition Database, or INFOODS.

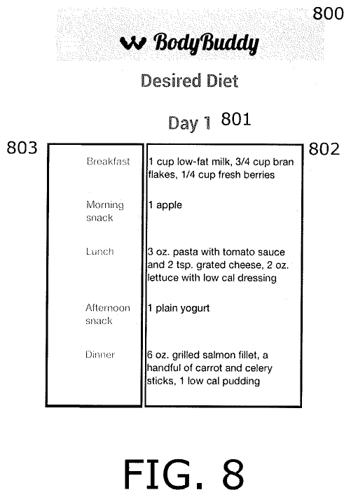

An example output screen for a diet plan is depicted in FIG. 8. FIG. 8 depicts an Diet Output Screen 800 with the Day 801 on which the modular diet plan elements should be consumed, the Meal Names 803, and the Foods 802 which should be consumed for their corresponding Meal Names 803.

As will be understood by those skilled in the art, FIG. 8 merely depicts an example of how a diet plan comprising at least one modular diet plan element may be described and communicated to a user. Other methods of describing and communicating a customized diet plan to a user are possible without departing from the scope of the invention.

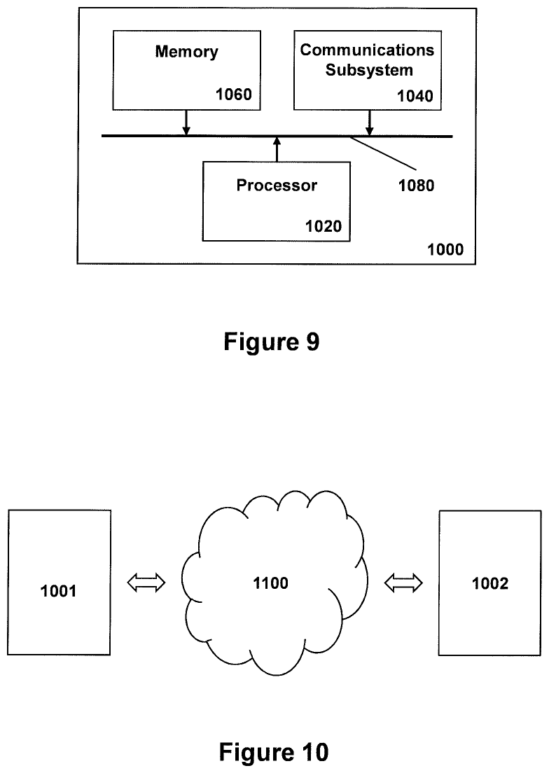

In FIG. 9, device 1000 includes a processor 1020 and a communications subsystem 1040, where the processor 1020 and communications subsystem 1040 cooperate to perform the methods of the embodiments described above.

Communications subsystem 1040 may, in some embodiments, comprise multiple subsystems, for example for different radio technologies.

It will be understood that processor 1020 is configured to execute programmable logic, which may be stored, along with data, on device 1000, and shown in the example of FIG. 9 as memory 1060. Memory 1060 can be any tangible, non-transitory computer readable storage medium. The computer readable storage medium may be a tangible and/or in transitory/non-transitory medium such as optical (e.g., CD, DVD, etc.), magnetic (e.g., tape), flash drive, hard drive, or any other suitable other memory known in the art.

Alternatively, or in addition to memory 1060, device 1000 may access data or programmable logic from an external storage medium, for example through communications subsystem 1040.

Communications subsystem 1040 allows device 1000 to communicate with other devices or network elements and may vary based on the type of communication being performed. Further, communications subsystem 1040 may comprise a plurality of communications technologies, including any suitable wired or wireless communications technologies.

In at least one embodiment, communications between the various elements of device 1000 may be through an internal bus 1080. However, it will be readily appreciated that other forms of communication are possible.

In at least one embodiment, communications subsystem 1040 allows device 1000 to communicate with other devices or network elements through a central distribution server over an electronic communication network. Communications subsystem 1040 may use one or more of a variety of communications types, including but not limited to cellular, satellite, Bluetooth™, Bluetooth™ Low Energy, Wi-Fi, wireless local area network (WLAN), near field communications (NFC), ZigBee, wired connections such as Ethernet or fiber, among any other suitable options that will be readily appreciated by the skilled person. As such, a communications subsystem 1040 for wireless communications will typically have one or more receivers and transmitters, as well as associated components such as one or more antenna elements, local oscillators (LOs), and may include a processing module such as a digital signal processor (DSP). As will be readily apparent to those skilled in the field of communications, the particular design of the communication subsystem 1040 will be dependent upon the electronic communication network or communication technology on which the sensor apparatus is intended to operate.

If communications subsystem 1040 operates over a cellular connection, a subscriber identity module (SIM) may be provided to allow such communications. It is contemplated that a SIM may be a physical card or may be virtual. In some embodiments, a SIM may also be referred to as a universal subscriber identity module (USIM), as merely an identity module (IM), or as an embedded Universal Integrated Circuit Card (eUICC), among other options.

With reference to FIG. 10, a diagram of a first user device 1001 in electronic communication with an analogous second device 1002 over an electronic communication network 1100 is shown. As discussed herein, it is contemplated that first user device and second user device are analogous to user device 1000 discussed above, and that electronic communication network 1100 can take a variety of forms as discussed herein. For example, first user device 1001 can be in electronic communication with an analogous second device 1002 by way of near field communication (NFC) protocols or alternatively may be in electronic communication through a fixed or wireless link through a central distribution server, such as the internet.

The embodiments described herein are intended to be illustrative of the present compositions and methods and are not intended to limit the scope of the present invention. Various modifications and changes consistent with the description as a whole and which are readily apparent to the person of skill in the art are intended to be included. The appended claims should not be limited by the specific embodiments set forth in the examples but should be given the broadest interpretation consistent with the description as a whole.

1. An electronic method of devising a diet plan and a fitness plan for a user over an electronic communication network, comprising the steps of:
a) an electronic device receiving baseline measurement data relating to the user before a period of training and diet activity;
b) the electronic device receiving fitness goals data relating to the user, the fitness goal associated with the baseline measurement data;
c) assembling a training plan for the user based on at least one the baseline measurement data and the fitness goals data, the training plan selected from at least one modular training plan element retrieved by the electronic device from a central database, each at least one modular training plan element associated with at least one of the baseline measurement data and the fitness goals data;
d) assembling a diet plan for the user based on at least one of the baseline measurement data and the user's fitness goals data, the diet plan selected from at least one modular diet plan element retrieved by the electronic device from a central database, each at least one modular diet plan element associated with at least one of the baseline measurement data and the fitness goals data;
e) the electronic device receiving behavioural data describing at least one of the user's diet behaviour and the user's training behaviour during the period of training and diet activity;
f) the electronic device receiving current measurement data relating to the user after the end of the period of training and diet activity;
g) determining the difference between the current measurement data and the baseline measurement data to generate a measurement data difference; and
h) comparing the measurement data difference to the fitness goal.

2. The method of claim 1, further comprising the steps of: if the measurement data difference does not satisfy the fitness goal,
i) the electronic device setting values of the baseline measurement data to values of the current measurement data;
j) the electronic device setting the training plan to be an updated training plan, the updated training plan selected from at least one modular training plan element
k) retrieved by the electronic device from a central database, each at least one modular training plan element associated with at least one of the baseline measurement data and the fitness goals data;
l) the electronic device setting the diet plan to be an updated diet plan selected from at least one modular diet plan element retrieved by the electronic device
m) from a central database, each at least one modular diet plan element associated with at least one of the baseline measurement data and the fitness goals data;
n) the electronic device setting the period of training and diet activity to be an upcoming period of training and diet activity;
o) the electronic device receiving updated behavioural data describing at least one
p) of the user's diet behaviour and the user's training behaviour during the updated period of training and diet activity;
q) the electronic device receiving updated current measurement data relating to the user after the end of the updated period of training and diet activity;
r) determining the difference between the updated current measurement data and s) the baseline measurement data to generate an updated measurement data difference; and
t) comparing the updated measurement data difference to the fitness goal.

3. The method of claim 2, further comprising the step of repeating steps i) to p) until the measurement data difference matches or exceeds the fitness goal.

4. The method of claim 1, wherein the at least one predetermined training plan module is retrieved based on exercise preferences of the user.

5. The method of claim 1, wherein at least one predetermined training plan module is retrieved based on which exercise equipment is available to the user.

6. The method of claim 1, wherein the at least one predetermined diet plan module is retrieved based on dietary needs or preferences of the user.

7. The method of claim 1, wherein the electronic device is a smart phone, a tablet computer, a laptop computer, wearable electronic device, or a desktop computer.

8. The method of claim 1, wherein the baseline measurement data and the current measurement data each comprise at least one of: age of the user; sex of the user; weight of the user; height of the user; and body dimensions of the user.

9. The method of claim 1, wherein the fitness goals comprise at least one of: improve definition of at least one muscle group; lose fat; lose weight; and gain muscle mass.

10. The method of claim 1, wherein the diet plan comprises at least one of: instructions for all meals during the period of training and diet activity; suggestions for types of foods and meals during the period of training and diet activity; suggestions for amounts of foods during the period of training and diet activity; suggestions for timing of meals during the period of training and diet activity; and instructions for vitamins and nutrients to eat during the period of training and diet activity.

11. The method of claim 1, wherein the training plan comprises at least one of: a specific exercise routine for the period of training and diet activity; suggested types of exercises for the period of training and diet activity; suggested amounts of time to spend performing exercises during the period of training and diet activity; suggested levels of intensity for exercises during the period of training and diet activity; and suggested frequency to perform exercises during the period of diet and training activity.

12. The method of claim 8, wherein the user's body dimensions are determined by analyzing visual data of the user using photogrammetry.

13. The method of claim 12, wherein photogrammetry is used to create an electronic three-dimensional model of the user.

14. The method of claim 13, wherein the electronic device displays the electronic three-dimensional model to the user and provides an interface which can be used to set fitness goals by interacting with parts of the electronic three-dimensional model.

15. The method of claim 12, wherein the visual data is obtained by at least one of: taking photographs of the user; and recording video of the user.

16. The method of claim 12, wherein the visual data is obtained by using a camera which is connected to the electronic device.

17. The method of claim 12, wherein the electronic device performs photogrammetry processing which determines the user's body dimensions.

18. The method of claim 1, wherein if the electronic device receives new fitness goals after the diet plan and the training plan have been created then the electronic device creates a new training plan and a new diet plan based on the new fitness goals.

19. The method of claim 1 wherein the electronic device analyzes the fitness goals, determines the probability of the user achieving the fitness goals, and, if the electronic device determines that the user has a higher probability of failing the fitness goals than achieving the fitness goals, the electronic device suggests new fitness goals which the user has a higher probability of achieving.

20. The method of claim 1, wherein assembling a training plan from at least one predetermined training plan module retrieved by the electronic device from a central database, further includes tracking which training plan modules most frequently satisfy fitness goals data in measurement data contexts and weighting frequently successful training plan modules to be selected more often in similar measurement data contexts.

21. The method of claim 1, wherein assembling a diet plan from at least one predetermined diet plan module retrieved by the electronic device from a central database, further includes tracking which diet plan modules most frequently satisfy fitness goals data in measurement data contexts and weighting frequently successful diet plan modules to be selected more often in similar measurement data contexts.
